(19) United States
(12) Patent Application Publication
(10) Pub. No.: US 2024/0216562 A1
(43) Pub. Date: Jul. 4, 2024

(54) NASAL SWAB APPARATUS AND METHOD FOR APPLYING A CLEANING SOLUTION

(71) Applicant: Polley Med, LLC, East Windsor, NJ (US)

(72) Inventors: John W. Polley, Maple City, MI (US); Sean Yeom, Cranbury, NJ (US)

(21) Appl. No.: 18/605,418

(22) Filed: Mar. 14, 2024

Related U.S. Application Data
(63) Continuation-in-part of application No. PCT/US2023/062895, filed on Feb. 20, 2023.
(60) Provisional application No. 63/268,315, filed on Feb. 22, 2022.

(51) Int. Cl. A61L 2/26; A61L 2/00; A61L 2/18

(52) U.S. Cl. CPC A61L 2/26; A61L 2/0088; A61L 2/18; A61L 2202/181

(57) ABSTRACT

A nasal swab kit for cleaning and sanitizing human nose nasal vestibules includes a pair of nasal swab devices, each of the devices having a body from which two spaced apart prongs extend, and each of the prongs having a free end to which a swab is attached. The swabs of a first of the devices are saturated with at least one cleaning ingredient selected from sterile water, glycerin, sodium chloride, sodium benzoate, PEG 60 hydrogenated castor oil, cocamidopropyl betaine and lavender oil. The swabs of a second of the devices are saturated with a sanitizing ingredient such as ethyl alcohol. The kit includes a first packet inside which the first device is sealed and a second packet inside which the second device is sealed wherein the packets are conjoined at a perforated seam.

### CROSS-REFERENCE TO RELATED APPLICATIONS

This application is a continuation-in-part of the co-pending international patent application Serial No. PCT/US2023/062895 filed Feb. 20, 2023. This application claims the benefit of U.S. provisional patent application Ser. No. 63/268,315 filed Feb. 22, 2022.

Incorporated by reference are U.S. provisional patent application Ser. No. 63/268,315 filed Feb. 22, 2022, PCT international application serial no. PCT/US2023/062895 filed Feb. 20, 2023, and U.S. design patent application Ser. No. 29/909,427 filed Aug. 4, 2023.

### FIELD OF THE INVENTION

This invention relates to nasal swabs and methods of using nasal swabs. More particularly, the invention is directed to a double prong nasal swabs with a columellar (Nasal, Alar, Nasal Rim, Nasal Opening, Nasal Vestibular, Vestibular Rim) guard and a nasal cleaning solution and a method for the cleaning, washing, prepping, decolonizing, and/or sterilizing of the human nose and nasal vestibules.

### BACKGROUND OF THE INVENTION

This section provides background information related to the present disclosure which is not necessarily prior art.

A benefit of the COVID-19 pandemic is an increase in both public and personal awareness of communicable diseases. Most importantly has been the increased awareness of the method of transmission of respiratory illnesses. Hygiene is of paramount importance, and primary emphasis has been on hand washing and the avoidance of hand to face contact. A clear understanding of the pathophysiology of the infectious process, for both viral and bacterial infections, is essential in formulating appropriate hygiene methodologies.

Numerous studies have clearly demonstrated that the pathway in which bacterial and viral respiratory infections are initially transmitted to humans is by way of the nasal cavity (nose) (Publications 1-9 identified below). 90% of inspiration (breathing) is through the nose. The front half or anterior portion of the nose is called the nasal vestibule. The nasal vestibules, in an adult, extend posteriorly approximately 2-½ cm (1 inch) from the nostril rims. The nasal vestibules are lined with desquamating epithelium or skin, the same skin as found throughout the rest of our bodies. The skin of the nasal vestibules is rich with hair and hair follicles, sweat glands, apocrine glands, and mucous glands. (Publications 10-12). The nasal vestibules with hair follicles and sticky mucus act as the major filter for the air that we breathe in. In vivo experiments utilizing laser Optic identification techniques have demonstrated that the anterior vestibule of the nose with its hair and mucus secretions can filter inspired particles greater than or equal to 0.5 μm. (Publications 13-17). Most bacteria range from 0.5 um-30 um. in diameter and plant spores and plant pollens range on average size from 10 to 100 μm in diameter. Most viruses in isolation range under 0.5 μm. (Publications 18,19). Most viruses do not transport in isolation but are enveloped in a water or dust droplet that range from 0.5 μm or greater. Viruses, pollens, and spores are themselves immobile, they must be physically transported past the nasal vestibule to the posterior nasal mucosa physically by inspiratory pressures and or by overwhelming the filter capacity of the nasal vestibule and then continuing posterior with further inspiratory pressure. It is well-recognized that the greater the quantity of allergens a patient is challenged with, the greater their symptoms of allergic rhinitis. While some bacteria can be expelled as a droplet, most bacteria are transported mechanically through hand to face contact (10,11,20,21)

Once trapped in the nasal vestibule viruses and bacteria can colonize by attaching to host nasal epithelial cells, replicate, and when reaching a critical mass (incubation period) invades further into the nasal and respiratory tract. Nasal vestibular epithelium and posterior nasal mucosal cells are abundant in angiotensin-converting enzyme 2 (Ace2) and transmembrane protease serine 2 (TMPRSS2), host proteins essential for the attachment to and infection of host target cells by SARS-CoV-2, influenza and other viruses (Publications 2-5, 22). The importance of the anterior nose or nasal vestibule in hosting the SARS-Co-V-2 virus is illustrated by the change in nasal swab techniques for testing for Covid-19. Initially in the pandemic, the nasal swab testing technique included deep penetration of the swab into the nasal cavity, far beyond the posterior border of the nasal vestibule. Current techniques swab only the nasal vestibule, not further posterior into the nasal mucosa (Publication 23, 24). The incubation period in the nasal vestibules from initial nasal contagion until infection for viruses varies between 3-14 days (Publications 3, 22).

This nasal pathophysiologic process of colonization, incubation, infection and transmission is true not only for viral infections but for bacterial infections as well. One third or more of the human population is a nasal carrier of staphylococcus aureus and numerous other gram positive and gram negative bacteria (Publications 25, 26). These bacteria are responsible for the majority of hospital and long term care facility acquired infections. They are also primarily responsible for post and peri-operative infections. Routine nasal vestibular hygiene has been demonstrated to reduce the incidence of these nosocomial infections (Publications 27-29).

Early therapeutic elimination of colonizing viruses and bacteria in the nasal cavity can significantly reduce the risk of person-to-person transmission as well as propagation of the infection to the lower respiratory tract and other organs.

With this understanding of the pathophysiology of the infectious process of viruses and bacteria we can develop a scientific approach to prophylactic hygiene. Early therapeutic elimination of colonizing viruses and bacteria in the nasal cavity can significantly reduce the risk of person-to-person transmission as well as propagation of the infection to the lower respiratory tract and other organs.

Nasal disinfection should be the key component of prophylactic hygiene following exposure to potential sources of airborne viruses and bacteria (crowds, airplanes, close public contact, known carriers, etc.). Few studies have examined this and these primarily reviewed mouth rinsing, nasal sprays, and nasal lavage techniques (Publications 30-33). The medical and nonprofessional literature lacks studies on how to prep or sterilize the nose. The reason is that published authors in this field have no experience on how to prep/sterilize/clean the nasal vestibules. What is needed is a simple, effective, well documented true surgically proven nasal cleaning and disinfecting technique with the appropriate utensil that can eradicate harmful bacteria, viruses, and allergic dander's in the nasal vestibule during the early colonization phase, prior to overt infection.

Bibliography of publications on pathophysiology and disinfectants:
1. Zou L, et al. SARS-CoV-2 viral load in upper respiratory specimens of infected patients. N Engl J Med 382(12), 1177-1179 2020
2. Gengler I, et al: Sinonasal pathophysiology of SAS-CoV-2 and COVID-19: A systematic review of the current evidence. Laryngoscope Investig Otolaryngol, Vol 5(3):pp 354-359, June 2020
3. Young B E, et al. Epidemiologic features and clinical course of patients infected with SARS-COV-2 in Singapore. JAMA. 2020.10.1001/jama.2020.3204
4. Gallo O. et al: The central role of the nasal microenvironment in the transmission, modulation, and clinical progression of SAS-CoV-2 infection. Mucosal Immunology, Vol 14, pp 305-316, 2021
5. Andersen, K G, et al. The proximal origin of SARS-CoV-2. Nat Med 26,450-452 2020
6. Zou L, et al. SARS-CoV-2 viral load in upper respiratory specimens of infected patients. N Eng J Med, https://doi.org/10.1056/NEJMc2001737 2020
7. Sungnak W. et al. SARS-CoV-2 entry factors are highly expressed in nasal epithelial cells together with innate immune genes. Nat Med 26, 681-687 2020
8. Hou Y, et al. Researchers map how the coronavirus infection travels through cells of nasal cavity and respiratory tract. Gillings School News, Epidemiology News Un N Carolina Gillings School Global Pub Health 2021
9. Rothe C, et al. Transmission of 2019-nCoV infection from an asymptomatic contact in Germany. N Eng J Med 2020;382:970-971
10. O'Neal R M, Beil Jr R J, Schlesinger J. Surgical anatomy of the nose. Otolatyngol Clin North Am. 1999 February;32(1):145-81 (PM: 10196443)
11. Beule A G. Physiology and pathophysiology of respiratory mucosa of the nose and paranasal sinuses. GMS current topics in otofhinolaryngology-head and neck surgery 2010, Vol 9 ISSN 1865
12. Cole P. Nasal and oral airflow resistors. Site, function, and assessment. Arch Otolaryngol Heas Neck Surg. 1992;118:790-793
13. Hatch T F, Gross P. Pulmonary deposition and retention of inhaled aerosols. NY, London Acedemic press. 1964
14. Xie X, et al. How far droplets can move in indoor environments-revisiting the Wells evaporation-falling curve. Indoor Air 2007; 17:211-225. DOI:10.1111
15. Zhou Y, et al. Particle size distribution and inhalation dose of liquids under selected operating conditions. Inhal Toxicol, 2007; 19:333-342. DOI:10.1080
16. Cole P. Nasal and oral airflow resistors. Site, function, and assessment. Arch Otolaryngol Heas Neck Surg. 1992;118:790-793
17. O'Neal R M, Beil Jr R J, Schlesinger J. Surgical anatomy of the nose. Otolatyngol Clin mNorth A. 1999 this February;32(1):145-81 (PM: 10196443)
18. Beule A G. Physiology and pathophysiology of respiratory mucosa of the nose and paranasal sinuses. GMS current topics in otofhinolaryngology-head and neck surgery 2010, Vol 9 ISSN 1865
19. Harrison) SC (1984) Structure of viruses. Soc Gen Microbiol Symp. 36, 29 Cambridge Un press, Cambride 1984
20. DeShazo R D, Corren J. Allergic rhinitis: clinical manifestations, epidemiology, and diagnosis. Wolters Kluwer UpToDate Inc. 2023 www.uptodate.com
21. Dykewicz M S, et al. Rhinitis 2020: A practice parameter update. J Allergy Clin Immunol 2020; 146:721
22. ZIEGLER C G, ET AL Sars-Cov-2 receptor E2 is an interferon-stimulated gene in human airway epithelial cells and is detected in specific cell subsets across tissues. Cell
23. Heikkinen T, et al. Nasal swab versus nasopharyngeal aspirate for isolation of respiratory viruses. J Clin Microbiol, 2022 November; 40(11):4337-4339 (PM 12409435)
24. Waris M E, et al. et al. Nasal swabs for detection of respiratory syncytial virus RNA. Arch Dis Child, 2007 November; 92(11):1046-1048 (PM 17626142)
25. Bode L G, et al. Preventing surgical-site infections in nasal carriers of Staph. Aureus. N Engl J Med 2010:362:9-17.
26. Wertheim H F, et al. The role of nasal carriage in Staph. Aureus infections. Lancet Infect Dis 2005:5:751-62
27. Steed L L, et al. Reduction of nasal Staph Aureus carriage in health care professionals by treatment with a nonantibiotic, alcohol-based nasal antiseptic. Amer J Inf Control 2014:42:841-6.
28. Huang S S, et al. Targeted vs universal decolonization to prevent ICU infection. N Engl J Med 2013:368:2255-65.
29. Septimus E J. Nasal decolonization: What antimicrobials are most effective prior to surgery? Amer J Inf Control 2019:47:A53-A57
30. European Rhinologic Society. Information for Rhinologists on COVID-19. https://www.europeanrhinologicsociety.org/id=2143 Accessed Mar. 30, 2020
31. American Academy of Otolaryngology-Head and Neck Surgery Academy SupportsCMS,OffersSpecificNasalPolicy https://www.entnet.org/content/academy-supports-cms-offers-specific-nasal-policy. Accessed Mar. 30, 2020
32. Stathis, C, et al: Review of the use of nasal and oral antiseptics during a global pandemic. Future Microbiol, 16(2):pp 119-130, January 2021
33. Kampf G, et al. Persistence of coronaviruses on inanimate surfaces and their inactivation with biocidal agents. J Hosp Infect 104(3), 246-251 2020

### BRIEF SUMMARY OF THE INVENTION

According an embodiment of the present invention, a “double prong/dual swab” nasal swab includes a columellar (Nasal, Alar, Nasal Rim, Nasal Opening, Nasal Vestibular, Vestibular Rim) guard or stop, and an antiseptic or disinfectant cleaning solution.

A complete nasal surgical prep/nasal decolonization/ nasal hygiene system, with instructions on use, is the goal of the nasal swab apparatus according to the invention. It offers the following advantages:
Professional (MD) surgically based nasal prep cleaning method
Easy to use, foolproof
Compact, lightweight, disposable, inexpensive, safe
Can be used anytime, anywhere, without social concern
No need for sinks, water, private space to use
Assures proper placement of the device only in the nasal vestibule
Does not require medical professional supervision for use (OTC)

A device according to the invention is a double prong swab which consists of consists of a handle and two prongs with a swab on each end of the prongs. The handle is for holding the device with human fingers or hands. The superior border of the handle between the two prongs acts as a nasal or columellar guard to prevent entry of the prongs too far into the nose. The columellar guard of the handle assures proper placement of the swabs, at the end of the prongs, only in the nasal vestibular skin and not higher into the nasal mucosa, or too shallow of placement in the nasal vestibules. The swab is for intranasal or nasal vestibular use or placement. Accurate anatomic placement of the swab(s) ensures thorough cleaning, scrubbing, prepping, disinfecting, and/or decolonizing of the entire nasal vestibule(s).

The device can be constructed from any type of material such as paper or paper product, plastic of any type, metal, synthetic product or paper. The device can be made of biodegradable or non-biodegradable materials. The device has attached to or as part of it a swab, pad, wipe, gauze, wad, foam, sponge, cushion, mop, bolster, and/or covering.

The device is an evidence based daily use nasal vestibular hygiene decolonization apparatus for performing a method and technique that safely and effectively cleans the nose the correct way. The method is a 2-step procedure which adheres to the surgically documented superiority of a 2-step, dual prep technique, for skin sanitation and decolonization. The device consists of two pre-saturated dual swab devices, with each double-pronged device packaged in a separate packet. Two packets, step 1 and step 2, each containing a separate presaturated dual swab device. The packets are conjoined as a single two piece packet with a perforated seam between the two packets which allows for easy separation of step 1 and step 2 packets if desired. Step 1 is the cleaning step with the dual swabs saturated with the ingredients: sterile water; glycerin; sodium chloride; sodium benzoate; PEG 60 hydrogenated castor oil; cocamidopropyl betaine; and lavender oil. Step 2 is the sanitizing step with the ingredients: 62% ethyl alcohol and 38% sterile water. The two step nasal decolonization/hygiene technique includes: Step 1 cleaning the nasal vestibular skin of carriage with the step 1 device; and Step 2 sanitizing a now clean nasal vestibular lining with the step 2 device. This method/technique avoids the cross and recontamination of other techniques and, for the first time, offers an evidence based surgical protocol for nasal decolonization. The unique dual swab design of the devices has a columellar guard, assuring proper placement only in the nasal vestibular skin, not higher up into the more fragile nasal mucosa. The device is portable, disposable, can be used anytime, anywhere discreetly without the need of medical assistance, running water, or other utensils or vesicle containing solutions.

The device can be packaged in, held in, delivered in, wrapped in, parceled in, a plastic or paper or metallic (or other synthetic based material) container with rip off or seal access. The package can contain antiseptic(s) that soak the swab(s) of the device in a various fashions. The package or kit can contain one, two, or more of the devices.

The device can be used to perform a method for the cleaning, washing, prepping, decolonizing and/or sterilizing of the human nose regardless of the sex or age of the person. The method can be used in medicine as a surgical prep/sterilization technique. The method can be used for commercial or public use enabling definitive nasal hygiene. The method can utilize alone or in any combination, FDA approved surgical prep antiseptics or disinfectants and/or moisturizing agents.

The nasal columellar guide is formed by the superior border of the handle between the two prongs.

The body and/or the prongs are constructed from a material selected from a paper, a paper product, a plastic, a metal and a synthetic product. The material can be biodegradable.

The swab is adapted to hold a quantity of an antiseptic or a disinfectant and to release the antiseptic or the disinfectant upon contact with a surface in the nasal vestibule of the human nose.

According to a first aspect of the invention, a method for cleaning and sanitizing two nasal vestibules of a human nose comprises the steps of: simultaneously cleaning a nasal vestibular lining of carriage in the two nasal vestibules using a first nasal swab device saturated with at least one cleaning ingredient; and simultaneously sanitizing the cleaned nasal vestibular linings using a second nasal swab device saturated with a cleaning ingredient. Each of the cleaning step and the sanitizing step is performed by inserting swabs of the respective first and second devices into the two nasal vestibules and placing the swabs against the linings, rotating the swabs seven times in a circular motion by moving along a circular horizontal path without rotating about a longitudinal axis of the respective device.

According to a second aspect of the invention, the first nasal swab device has a pair of swabs saturated with sterile water, glycerin, sodium chloride, sodium benzoate, PEG 60 hydrogenated castor oil, cocamidopropyl betaine and lavender oil and the second nasal swab device has a pair of swabs saturated with 38% sterile water and 62% ethyl alcohol.

According to a third aspect of the invention, a nasal swab kit for cleaning and sanitizing human nose nasal vestibules comprises: a pair of nasal swab devices, each of the devices having a body from which two spaced apart prongs extend, and each of the prongs having a free end to which a swab is attached; wherein the swabs of a first of the devices are saturated with at least one cleaning ingredient and the swabs of a second of the devices are saturated with a sanitizing ingredient; a first packet inside which the first device is sealed and a second packet inside which the second device is sealed; wherein the first packet and the second packet are conjoined at a perforated seam; and wherein the first packet and the second packet each have a notch formed in an edge thereof providing starting points for tearing open the packets.

According to a fourth aspect of the invention, the at least one cleaning ingredient is selected from sterile water, glycerin, sodium chloride, sodium benzoate, PEG 60 hydrogenated castor oil, cocamidopropyl betaine and lavender oil and the sanitizing ingredient is ethyl alcohol.

According to a fifth aspect of the invention, the swabs of the first device are saturated with sterile water, glycerin, sodium chloride, sodium benzoate, PEG 60 hydrogenated castor oil, cocamidopropyl betaine and lavender oil.

According to a sixth aspect of the invention, the swabs of the second device are saturated with 62% ethyl alcohol and 38% sterile water.

According to a seventh aspect of the invention, the first packet is identified by a “step 1 clean” label on an outside surface thereof and the second packet is identified by a “step 2 sanitize” label on an outside surface thereof.

### BRIEF DESCRIPTION OF THE SEVERAL VIEWS OF THE DRAWING

The above as well as other advantages of the invention will become readily apparent to those skilled in the art from the following detailed description of a preferred embodiment when considered in the light of the accompanying drawings in which:

FIG. 1 is a front view of a nasal swab device according to the invention;

FIG. 2 is a front view of alternate embodiments of the nasal swab device shown in FIG. 1;

FIG. 3 is a schematic view a nasal swab kit including two nasal swab devices according to the invention; and

FIG. 4 is a flow diagram of a method for using the nasal swab kit of FIG. 3 according to the invention.

### DETAILED DESCRIPTION OF THE INVENTION

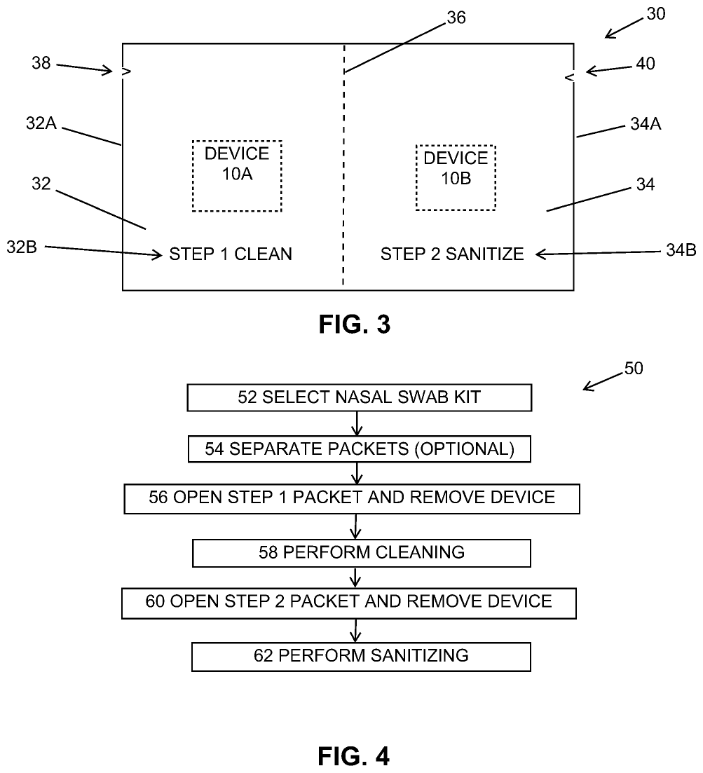

Various alternative embodiments of the invention are disclosed in detail in the U.S. provisional patent application Ser. No. 63/268,315 filed Feb. 22, 2022 and the international patent application Serial No. PCT/US2023/062895 filed Feb. 20, 2023. For example, see FIGS. 3-10 of the international application.

The above as well as other advantages of the present invention will become readily apparent to those skilled in the art from the following detailed description of a preferred embodiment when considered in the light of the accompanying drawings. The following detailed description and included drawings describe and illustrate various exemplary embodiments of the invention. The description and drawings serve to enable one skilled in the art to make and use the invention, and are not intended to limit the scope of the invention in any manner. In respect of the methods disclosed, the steps presented are exemplary in nature, and thus, the order of the steps is not necessary or critical.

More particularly, the invention is directed to single and multiple prong nasal swab devices with a columellar guard, a nasal cleaning solution, and a method for the cleaning, washing, prepping and/or sterilizing of the human nose nasal vestibules.

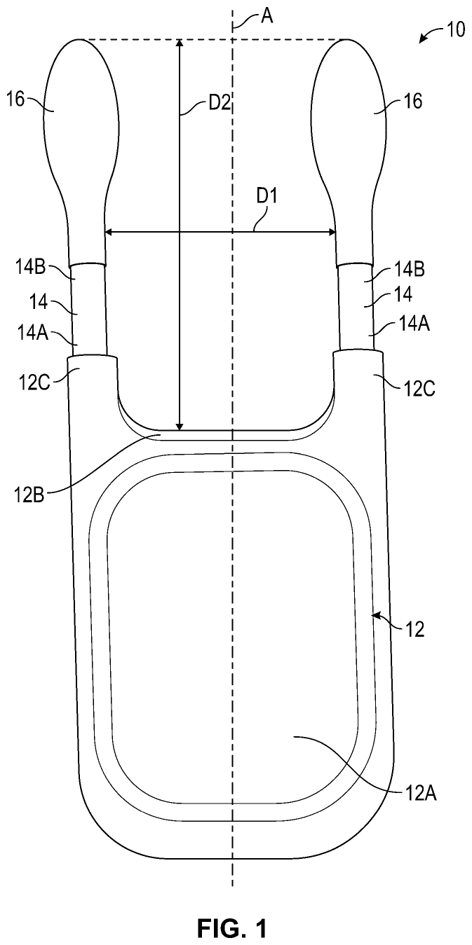

Shown in FIG. 1 is front side of a first embodiment of the nasal swab device 10 according to the invention. The rear side (not shown) can be identical. An elongated body 12 has a longitudinal axis A and is shaped as a handle or stem to be grasped between a thumb and a forefinger of a human hand (not shown). The body 12 can have central depressions 12A formed on opposite sides thereof as gripping aids. An attachment edge 12B of the body 12 has a pair of prongs or arms 14 extending therefrom generally parallel to the longitudinal axis A. The prongs 14 are spaced apart at a predetermined distance D1 to permit insertion into the nostrils of a human nose. Each of the prongs 14 has an attachment end 14A connected to the body 12 at the attachment edge 12B. The prongs 14 can be formed integral with the body 12. In the alternative, the prongs 14 can be separate parts with the attachment ends 14A either attached directly to the edge 12B or inserted into receptacles 12C extending from the edge 12B as shown in FIG. 1. When inserted in the receptacles 12C, the prongs 14 can be fixed or can be removable for replacement. Although the prongs 14 are shown as being of approximately the same length, they could be formed with different lengths.

Each of the prongs 14 has a free end 14B opposite the attachment end 14A. Each of the free ends 14B has an associated swab 16 attached thereto. Thus, the nasal swab device or apparatus 10 can be engaged by a human hand at the body 12 to position the swabs 16 simultaneously into separate ones of the nasal vestibules of a human nose. The portion of the attachment edge 12B that extends transversely between the attachment ends 14A functions as a nasal columellar guide, guard or stop. The nasal columellar guide limits an insertion depth D2 of the prongs 14 and swabs 16 into the nasal vestibules.

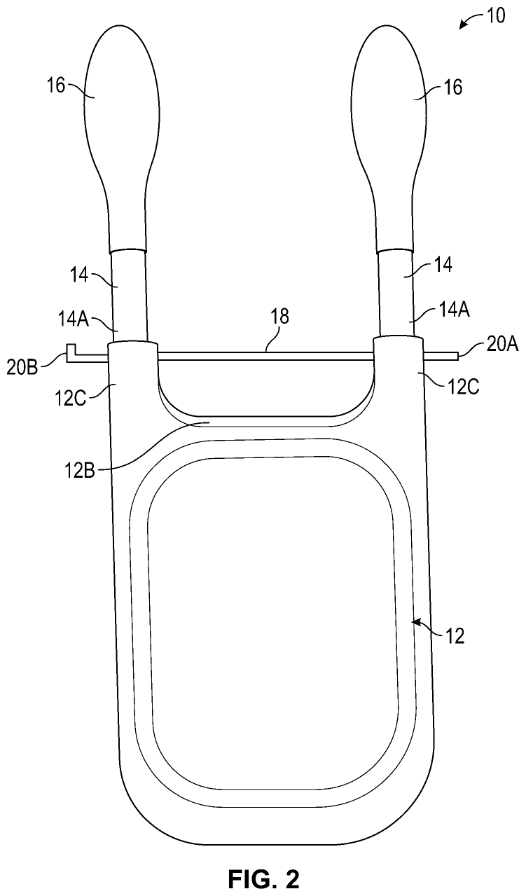

FIG. 2 shows two variations or alternate embodiments of the two prong nasal swab device 10 shown in FIG. 1. In a first variation, instead of the portion of the attachment edge 12B that extends transversely between the attachment ends 14A functioning as the nasal columellar guide, guard or stop, a separate nasal columellar guide 18 is spaced from the attachment edge 12B and extends between the receptacles 12C. In a second variation, a nasal alar guide, guard, stop is provided at each of the receptacles 12C. A nasal alar guide 20A extends from the right side receptacle 12C transverse to a longitudinal axis of the adjacent prong 14. A nasal alar guide 20B extends from the right side receptacle 12C transverse to a longitudinal axis of the adjacent prong 14. The guide 20B has an L-shape or could be curved. The nasal swab device 10 can be provided with any one or more of the guides 18, 20A and 20B.

The device 10 is used as a nasal vestibular hygiene decolonization apparatus for performing a method and technique that safely and effectively cleans the nose the correct way. The method is a 2-step procedure which adheres to the surgically documented superiority of a 2-step, dual prep technique, for skin sanitation and decolonization. The method can be performed using two pre-saturated double prong devices 10. Step 1 is the cleaning step with a first double prong device 10 having the two swabs 16 saturated with the cleaning ingredients: sterile water; glycerin; sodium chloride; sodium benzoate; PEG 60 hydrogenated castor oil; cocamidopropyl betaine; and lavender oil. Step 2 is the sanitizing step with a second double prong device 10 having the two swabs 16 saturated with the sanitizing ingredients: 62% ethyl alcohol and 38% sterile water. The 2-step nasal decolonization/hygiene technique includes: Step 1 cleaning the nasal vestibular skin of carriage with the step 1 device 10; and Step 2 sanitizing a now clean nasal vestibular lining with the step 2 device 10. This method/technique avoids the cross contamination and recontamination of other techniques and, for the first time, offers an evidence based surgical protocol for nasal decolonization. The unique dual swab design of the device 10 has a columellar guard 12B, 18, assuring proper placement only in the nasal vestibular skin, not higher up into the more fragile nasal mucosa. The device 10 is portable, disposable, can be used anytime, anywhere discreetly without the need of medical assistance, running water, or other utensils or vesicle containing solutions.

The step 1 device and the step 2 device can be provided as presaturated double prong devices 10 each in a separate sealed packet. As shown in FIG. 3, a nasal swab kit 30 includes a step 1 packet 32 releasably connected to a step 2 packet 34 along a perforated seam or tear line 36 at adjacent vertical edges of the packets. The perforated seam 36 allows for easy separation of the step 1 packet 32 from the step 2 packet 34 if desired. Sealed in the step 1 packet 32 is a step 1 device 10A having each swab presaturated with the cleaning ingredients described above. A notch 38 is formed in a packet edge 32A opposite the perforated seam 36 as a starting point for tearing open the packet 32. Sealed in the step 2 packet 34 is a step 2 device 10B having each swab presaturated with the sanitizing ingredients described above. A notch 40 is formed in a packet edge 34A opposite the perforated seam 36 as a starting point for tearing open the packet 34. The step 1 packet 32 is identified by a “STEP 1 CLEAN” label 32B on the outer surface. The step 2 packet 34 is identified by a “STEP 2 SANITIZE” label 34B on the outer surface.

The method steps for performing the 2-step nasal decolonization/hygiene technique according to the invention are shown in FIG. 4. The method 50 includes a first step 52 of selecting the nasal swab kit 30. In an optional second step 54, the conjoined packets 32, 34 can be separated at the perforated seam 36 if desired. In a third step 56, the step 1 packet 32 is opened by tearing horizontally starting at the notch 38 and the device 10A is removed from the packet. In a fourth step 58 the simultaneous cleaning of the nasal vestibular skin of carriage with the step 1 device 10A is performed. A preferred technique for using the devices 10A, 10B is to hold the handle 12 between the thumb and index finger of your hand. Then insert both swabs 16, one into each nostril, placing them against the nostril skin. The device 10A is then rotated seven times in a circular motion by moving the device along a circular horizontal path without rotating the device about the longitudinal axis A. Now the device 10A can be discarded.

In a fifth step 60, the step 2 packet 34 is opened by tearing horizontally starting at the notch 40 and the device 10B is removed from the packet. In a sixth step 62 the simultaneous sanitizing of a now clean nasal vestibular lining with the step 2 device 10B is performed. The handle 12 is held as described above. Then both swabs 16 are inserted, one into each nostril, placing them against the nostril skin. The device 10B is then rotated seven times in a circular motion by moving the device along a circular horizontal path without rotating the device about the longitudinal axis A. Now the device 10B can be discarded.

The cleaning step involves using the double prong device 10, 10A with the dual swabs 16 saturated with the ingredients: sterile water; glycerin; sodium chloride; sodium benzoate; PEG 60 hydrogenated castor oil; cocamidopropyl betaine; and lavender oil. The sanitizing step 2 involves using the double prong device 10, 10B with the dual swabs 16 with the ingredients: 62% ethyl alcohol and 38% sterile water. The 2-step nasal decolonization/hygiene technique includes: Step 1 cleaning the nasal vestibular skin of carriage with the step 1 device; and Step 2 sanitizing a now clean nasal vestibular lining with the step 2 device. This method/technique avoids the cross and recontamination of other techniques and, for the first time, offers an evidence based surgical protocol for nasal decolonization. The unique dual swab design of the devices has a columellar guard, assuring proper placement only in the nasal vestibular skin, not higher up into the more fragile nasal mucosa. The device is portable, disposable, can be used anytime, anywhere discreetly without the need of medical assistance, running water, or other utensils or vesicle containing solutions.

The nasal swab device can be packaged in, held in, delivered in, wrapped in, parceled in, a plastic or paper or metallic (or other synthetic based material) container/packet with tear off or seal access. The packet can contain antiseptic(s) that soak the swab(s) of the device in a various fashions. The package or kit according to the invention can contain one, two, or more of the nasal swab devices each sealed in a separate packet.

In accordance with the provisions of the patent statutes, the invention has been described in what is considered to represent its preferred embodiment. However, it should be noted that the invention can be practiced otherwise than as specifically illustrated and described without departing from its spirit or scope.

1. A nasal swab kit for cleaning and sanitizing human nose nasal vestibules, the nasal swab kit comprising:
a pair of nasal swab devices, each of the devices having a body from which two spaced apart prongs extend, and each of the prongs having a free end to which a swab is attached;
wherein the swabs of a first of the devices are saturated with at least one cleaning ingredient and the swabs of a second of the devices are saturated with a sanitizing ingredient; and
a first packet inside which the first device is sealed and a second packet inside which the second device is sealed.

2. The nasal swab kit according to claim 1 wherein the first packet and the second packet each have a notch formed in an edge thereof providing starting points for tearing open the packets.

3. The nasal swab kit according to claim 1 wherein the first packet and the second packet are conjoined at a perforated seam.

4. The nasal swab kit according to claim 1 wherein the at least one cleaning ingredient is selected from sterile water, glycerin, sodium chloride, sodium benzoate, PEG 60 hydrogenated castor oil, cocamidopropyl betaine and lavender oil.

5. The nasal swab kit according to claim 1 wherein the swabs of the first device are saturated with sterile water, glycerin, sodium chloride, sodium benzoate, PEG 60 hydrogenated castor oil, cocamidopropyl betaine and lavender oil.

6. The nasal swab kit according to claim 1 wherein the sanitizing ingredient is ethyl alcohol.

7. The nasal swab kit according to claim 1 wherein the swabs of the second device are saturated with 62% ethyl alcohol and 38% sterile water.

8. The nasal swab kit according to claim 1 wherein the first packet and the second packet are constructed from a material selected from a paper, a paper product, a plastic, a metal and a synthetic product.

9. The nasal swab kit according to claim 8 wherein the material is biodegradable.

10. The nasal swab kit according to claim 1 wherein the swabs are made of an absorbent material including any of cotton or other natural product, foam, sponge, synthetic fiber and gauze.

11. The nasal swab kit according to claim 1 wherein each of the devices includes a nasal columellar guard that extends transversely to a direction in which the two prongs extend, the nasal columellar guard being an edge of the body or a guide extending between receptacles extending from the body.

12. The nasal swab kit according to claim 1 wherein the first packet is identified by a “step 1 clean” label on an outside surface thereof and the second packet is identified by a “step 2 sanitize” label on an outside surface thereof.

13. A method for cleaning and sanitizing two nasal vestibules of a human nose, the method comprising the steps of:
simultaneously cleaning a nasal vestibular lining of carriage in the two nasal vestibules using a first nasal swab device saturated with at least one cleaning ingredient; and
simultaneously sanitizing the cleaned nasal vestibular linings using a second nasal swab device saturated with a sanitizing ingredient.

14. The method according to claim 13 wherein each of the cleaning step and the sanitizing step is performed by inserting swabs of the respective first and second devices into the two nasal vestibules and placing the swabs against the linings, rotating the swabs seven times in a circular motion by moving along a circular horizontal path without rotating about a longitudinal axis of the respective device.

15. The method according to claim 13 wherein the first nasal swab device has a pair of swabs saturated with sterile water, glycerin, sodium chloride, sodium benzoate, PEG 60 hydrogenated castor oil, cocamidopropyl betaine and lavender oil.

16. The method according to claim 13 wherein the second nasal swab device has a pair of swabs saturated with 38% sterile water and 62% ethyl alcohol.

17. A nasal swab kit for cleaning and sanitizing human nose nasal vestibules, the nasal swab kit comprising:
a pair of nasal swab devices, each of the devices having a body from which two spaced apart prongs extend, and each of the prongs having a free end to which a swab is attached;
wherein the swabs of a first of the devices are saturated with at least one cleaning ingredient and the swabs of a second of the devices are saturated with a sanitizing ingredient;
a first packet inside which the first device is sealed and a second packet inside which the second device is sealed;
wherein the first packet and the second packet are conjoined at a perforated seam; and
wherein the first packet and the second packet each have a notch formed in an edge thereof providing starting points for tearing open the packets.

18. The nasal swab kit according to claim 13 wherein the at least one cleaning ingredient is selected from sterile water, glycerin, sodium chloride, sodium benzoate, PEG 60 hydrogenated castor oil, cocamidopropyl betaine and lavender oil and the sanitizing ingredient is ethyl alcohol.

19. The nasal swab kit according to claim 13 wherein the swabs of the first device are saturated with sterile water, glycerin, sodium chloride, sodium benzoate, PEG 60 hydrogenated castor oil, cocamidopropyl betaine and lavender oil.

20. The nasal swab kit according to claim 13 wherein the swabs of the second device are saturated with 62% ethyl alcohol and 38% sterile water.

21. The nasal swab kit according to claim 13 wherein the first packet is identified by a “step 1 clean” label on an outside surface thereof and the second packet is identified by a “step 2 sanitize” label on an outside surface thereof.
